(19) United States
(12) Patent Application Publication
(10) Pub. No.: US 2010/0273266 A1
(43) Pub. Date: Oct. 28, 2010

(54) TARGET BIOMATERIAL DETECTING KIT AND METHOD OF DETECTING TARGET BIOMATERIAL

(75) Inventors: Jong-Cheol Hong, Daejeon (KR); Wan-Joong Kim, Gyeonggi-do (KR); Kyung-Hyun Kim, Daejeon (KR); Hyun-Sung Ko, Daejeon (KR); Chul Huh, Daejeon (KR); Gun Yong Sung, Daejeon (KR); Sun-Hee Park, Daejeon (KR)

Correspondence Address: AMPACC Law Group, 3500 188th Street S.W., Suite 103, Lynnwood, WA, 98037, US

(73) Assignee: ELECTRONICS AND TELECOMMUNICATIONS RESEARCH INSTITUTE, Daejeon (KR)

(21) Appl. No.: 12/747,504

(22) PCT Filed: Oct. 29, 2008
(86) PCT No.: PCT/KR2008/006362

§ 371 Date: Jun. 10, 2010

(30) Foreign Application Priority Data
Dec. 11, 2007 (KR) 10-2007-0128222

(51) Int. Cl. G01N 33/20

(52) U.S. Cl. 436/72; 436/81; 436/83; 436/73; 977/773

(57) ABSTRACT

Provided are a target biomaterial detecting kit and a method of detecting the target biomaterial. The target biomaterial detecting kit includes a guided mode resonance filter comprising a substrate transmitting or reflecting light, a grating layer formed on the substrate, and a capture layer formed on the grating layer to capture a target biomaterial; and a nano complex comprising a nanoparticle head and a connection tail. Therefore, the wavelength peak of a reflection/transmission spectrum of light coming from the guided mode resonance filter can be largely shifted, and thus the presence and quantity of a target biomaterial can be easily detected. Moreover, although the amount of the target biomaterial is small, the target biomaterial can be reliably detected.

### TECHNICAL FIELD

This application claims the benefit of Korean Patent Application No. 10-2007-0128222, filed on Dec. 11, 2007, in the Korean Intellectual Property Office, the disclosure of which is incorporated herein in its entirety by reference.

The present invention relates to a target biomaterial detecting kit and a method of detecting a target biomaterial, and more particularly, to a target biomaterial detecting kit capable of increasing the displacement of a wavelength peak of a reflection/transmission spectrum of light coming from a guided mode resonance filter, so as to easily and reliably detect the presence and quantity of a target biomaterial even when the amount of the target biomaterial is small, and a method of detecting a target biomaterial. The work was supported by the IT R&D program of MIC/IITA [2006-S-007-02, Ubiquitous Health Monitoring Module and System Development].

### BACKGROUND ART

Biosensors are used to detect the presences and quantities of biomaterials directly or indirectly related to vital activities, for example, materials such as enzymes, nucleotides, proteins, and cells. Such biosensors are used in a variety of fields such as gene expression, disease diagnosis, new drug development, and environmental monitoring. In disease diagnosis, the observation of phenomena such as color development and fluorescence caused by enzyme reactions is a commonly followed method of detecting a disease. In a recently developed immunoassay method, immune reactions between antigens and antibodies are used to diagnose diseases. In the immunoassay method, a label biosensor can be used to detect target antigens using antibodies marked with a radioisotope or a fluorescent material, and measure the amount of target antigens based on the intensity of radiation or fluorescence. However, since the immunoassay method using the label biosensor requires marked antibodies, its sample preparation process is complex. To address this problem, optical biosensors have been developed as label-free biosensors, and some examples of the optical biosensors include surface plasmon resonance biosensors, total internal reflection ellipsometry biosensors, and waveguide biosensors.

A resonant reflection biosensor, a kind of optical biosensor, is used to detect a target biomaterial by analyzing the peaks of reflection spectrums of light passing through a guided mode resonance filter (a diffraction grating functioning as a high refractive-index waveguide). The reflection spectrums measured from light diffracted by the diffraction grating and coupled to light propagating along the high refractive-index waveguide have a narrow line width. Therefore, the resonant reflection biosensor has a high sensitivity. In a current manufacturing method of the resonant reflection biosensor, the guided mode resonance filter is manufactured as follows: a diffraction grating is formed on a glass or polymer substrate by nano imprinting; a highly refractive index material such as SiNₓ or TiO₂ is applied to the diffraction grating so as to coat the diffraction grating; and a capture material capable of biochemically trapping a target biomaterial is fixed to the diffracting grating. When a target biomaterial, contained in a sample solution, is trapped by the capture material due to immune reactions or complementary binding, the refractive index and thickness of a layer of the capture material vary. Therefore, the target material can be identified by detecting and analyzing the variations of the refractive index and thickness using peak wavelengths of reflection spectrums. Factors that affect the variations of the refractive index include the molecular size of the target biomaterial, the molecular size of the coupled target material and the capture material, the refractive index of the target biomaterial, the refractive index of the sample solution containing the target material. In general, such factors result in a small variation in the refractive index, and thus the difference between peak wavelengths before and after coupling of a target biomaterial and a capture material is small at about 1 Å (10⁻¹⁰ m). Thus, it is difficult to detect such a small wavelength difference using a typical spectroscope.

### DISCLOSURE OF INVENTION

### Technical Problem

The present invention provides a target biomaterial detecting kit capable of increasing the displacement of a wavelength peak of a reflection/transmission spectrum of light coming from a guided mode resonance filter, so as to easily and reliably detect the presence and quantity of a target biomaterial even when the amount of the target biomaterial is small.

The present invention also provides a method of detecting a target biomaterial. In the method, the displacement of a wavelength peak of a reflection/transmission spectrum of light coming from a guided mode resonance filter is largely increased, so as to easily and reliably detect the presence and quantity of a target biomaterial even when the amount of the target biomaterial is small.

### Technical Solution

According to an aspect of the present invention, there is provided a target biomaterial detecting kit including a guided mode resonance filter and a nano complex. The guided mode resonance filter may include a substrate transmitting or reflecting light, a grating layer formed on the substrate, and a capture layer formed on the grating layer to capture a target biomaterial. The nano complex may include a nanoparticle head and a connection tail. The nanoparticle head may be formed of a metal oxide, a metal nitride, a metal sulfide, a silicon oxide, a silicon nitride, or a silicon sulfide. The connection tail may be connected to the nanoparticle head and be capable of biochemically coupling to the target biomaterial.

The nanoparticle head may be formed of a zinc sulfide, TiO₂, an indium tin oxide, an indium zinc oxide, a tantalum oxide, a silicon nitride, or a silicon oxide. The nanoparticle head may have a size in a range of about 1 nm to about 60 nm.

The connection tail may couple with the target biomaterial in a biochemical way such as an antigen-antibody reaction. The connection tail may be connected to the nanoparticle head using ethoxy silane based-hydrocarbon having three to nine carbon atoms or methoxy silane based-hydrocarbon having two to nine carbon atoms.

According to another aspect of the present invention, there is provided a method of detecting a target biomaterial, the method including: providing a guided mode resonance filter, bringing the capture layer of the guided mode resonance filter into contact with a sample containing a target biomaterial; and bringing a nano complex into contact with the target biomaterial. The bringing of the capture layer into contact with the sample may be performed before, during, or after the brining of the nano complex into contact with the target biomaterial.

### ADVANTAGEOUS EFFECTS

According to the present invention, the wavelength peak of a reflection/transmission spectrum of light coming from the guided mode resonance filter can be largely shifted, and thus the presence and quantity of a target biomaterial can be easily detected. Moreover, although the amount of the target biomaterial is small, the target biomaterial can be reliably detected.

### DESCRIPTION OF DRAWINGS

The above and other features and advantages of the present invention will become more apparent by describing in detail exemplary embodiments thereof with reference to the attached drawings in which:

FIG. 1 is a schematic view illustrating a target biomaterial detecting kit according to an embodiment of the present invention;

FIG. 2 is an enlarged view of portion A of FIG. 1;

FIG. 3 is a graph showing displacements of a peak wavelength according to an embodiment of the present invention;

FIG. 4 is a view illustrating a method of fabricating a nano complex, according to an embodiment of the present invention;

FIGS. 5A through 5C are schematic views illustrating reactions between a target biomaterial and a nano complex for different reaction sequences, according to embodiments of the present invention;

FIGS. 6A and 6B are graphs showing wavelength peaks for differently-sized nanoparticle heads of a nano complex; and

FIG. 7 is a graph illustrating a relationship between wavelength peak displacement and nanoparticle head size.

### BEST MODE

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. In the drawings, like reference numerals in the drawings denote like elements, and the thicknesses of layers and regions are exaggerated for clarity.

The present invention provides a target biomaterial detecting kit including a guided mode resonance filter and a nano complex.

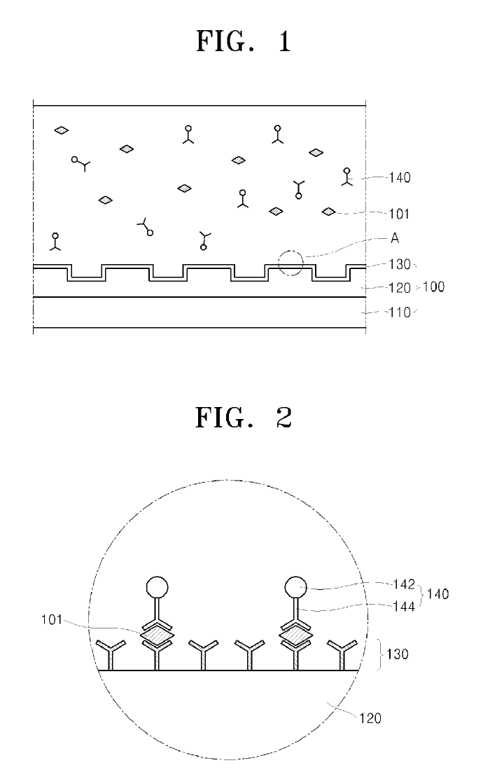

FIG. 1 is a schematic view illustrating a target biomaterial detecting kit according to an embodiment of the present invention, and FIG. 2 is an enlarged view of portion A of FIG. 1. Referring to FIGS. 1 and 2, the target biomaterial detecting kit includes a guided mode resonance filter 100 and a nano complex 140.

The guided mode resonance filter 100 includes a substrate 110, a grating layer 120, and a capture layer 130. The substrate 110 transmits or reflects light. The grating layer 120 is formed on the substrate 110, and the capture layer 130 is formed on the grating layer 120 in order to trap a target biomaterial 101.

The substrate 110 may be formed of glass or polymer. Alternatively, the substrate 110 may be formed of a low refractive index material such as MgF₂ (refractive index n=1.35), fluorinated ethylene propylene (FEP) copolymer (n=1.34), and polytetrafluoroethylene (PTFE, n=1.35).

The grating layer 120 is a diffraction grating having a refractive index greater than that of the substrate 110 or a sample solution. The grating layer 120 may be formed by a method such as etching and nano imprinting according to a material used for forming the grating layer 120. For example, the grating layer 120 may be formed of polymer resin, SiO₂, SiNₓ, or TiO₂. Examples of the polymer resin include polypropylene, polystyrene, and polycarbonate. However, the grating layer 120 can be formed of other materials.

Properties of the grating layer 120 such as a pitch and a refractive index are parameters determining the resonance wavelength of the guided mode resonance filter 100. In general, when the grating layer 120 has a pitch smaller than the wavelength of light incident onto the grating layer 120, primary resonant reflection may occur at the grating layer 120. When the grating layer 120 has a pitch greater than the wavelength of light incident onto the grating layer 120, second order or higher order diffraction may occur at the grating layer 120. In the guided mode resonance filter 100, light diffracted by the grating layer 120 couples with light propagating along a high refractive-index region (a kind of optical waveguide) surrounded by low reflective materials (the substrate 110 and a sample solution). The resonant reflection spectrum of the light passing through the guided mode resonance filter 100 has a narrow line width. For this, the guided mode resonance filter 100 has a narrow line width. The guided mode resonance filter 100 can be used in various optical systems and bisensors requiring a narrow line width filter.

The capture layer 130 is fixed to a surface of the grating layer 120 in order to biochemically couple with a specific target biomaterial such as the target biomaterial 101. Well-known methods can be used to fix the capture layer 130 to the grating layer 120. Thus, detailed descriptions of such methods will be omitted.

The capture layer 130 can be formed of a predetermined material depending on the kind of the target biomaterial 101. For example, when the target biomaterial 101 is a nucleic acid, such as DNA or RNA, a nucleic acid having a base sequence complementary to the base sequence of the target biomaterial 101 may be fixed onto the surface of the guided mode resonance filter 100 as the capture layer 130. Alternatively, when the target biomaterial 101 is immune antigens, proteins, or cells, corresponding antibodies may be fixed onto the surface of the guided mode resonance filter 100 as the capture layer 130.

Optionally, a material layer (not shown) having a high refractive index can be disposed between the grating layer 120 and the capture layer 130 to increase the effective refractive index of the guided mode resonance filter 100. In this case, the waveguide and resonance efficiencies of the guided mode resonance filter 100 may increase, and thus desired reflection spectrum peak characteristics can be easily attained.

When the capture layer 130, fixed to the surface of the guided mode resonance filter 100, biochemically couples with the target biomaterial 101, the optical characteristics of the capture layer 130 vary. Therefore, the presence and quantity of the target biomaterial 101 can be detected by measuring the variation in the optical characteristics of the capture layer 130. The variation in the optical characteristics of the capture layer 130 can be increased when the nano complex 140 is attached to the target biomaterial 101 coupled to the capture layer 130. That is, in this case, the displacement of a wavelength peak of the guided mode resonance filter 100 increases, and thus the target biomaterial 101 can be detected more sensitively.

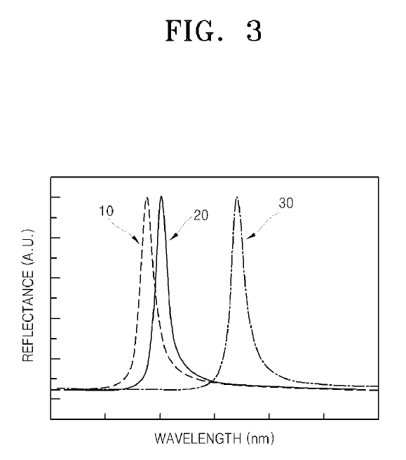

FIG. 3 is a graph showing displacements of a peak wavelength so as to compare the case where the nano complex 140 is not coupled to the target biomaterial 101 with the case where the nano complex 140 is coupled to the target biomaterial 101, according to an embodiment of the present invention.

Referring to FIG. 3, a reference wavelength peak 10 was measured before the target biomaterial 101 was coupled to the capture layer 130, and a wavelength peak 20 was measured after the target biomaterial 101 was coupled to the capture layer 130. Since the distance between the reference wavelength peak 10 and the wavelength peak 20 is small, it is difficult to detect the presence of the target biomaterial 101 based on the distance between the wavelength peak 10 and the wavelength peak 20.

A wavelength peak 30 was measured after the nano complex 140 was attached to the target biomaterial 101 coupled to the capture layer 130. Since the distance between the reference wavelength peak 10 and the wavelength peak 30 is large, it is easy to detect the presence of the target biomaterial 101. The displacement of a wavelength peak is proportional to the size and refractive index of molecules of the nano complex 140.

Each molecule of the nano complex 140 can be divided into two parts: a nanoparticle head 142 and a connection tail 144.

The nanoparticle head 142 can be formed of a metal oxide, a metal nitride, or a metal sulfide. Alternatively, the nanoparticle head 142 can be formed of a silicon oxide, a silicon nitride, or a silicon sulfide. Specifically, the nanoparticle head 142 can be formed of a zinc sulfide, TiO₂, an indium tin oxide, an indium zinc oxide, a tantalum oxide, a silicon nitride, or a silicon oxide. Other materials having high transmittance and high refractive index can be used to form the nanoparticle head 142.

The size of the nanoparticle head 142 may range from about 1 nm to about 100 nm. When the grating layer 120 has a small pitch of about 100 nm, the size of the nanoparticle head 142 may range from about 1 nm to about 60 nm. The sensitivity of the guided mode resonance filter 100 is proportional to the size and refractive index of the nanoparticle head 142. Here, the size of the nanoparticle head 142 is defined as the distance between the two most distant points of the nanoparticle head 142.

The connection tail 144 may be formed of a material capable of biochemically coupling with the target biomaterial 101. The biochemical coupling may be antigen-antibody coupling. If the target biomaterial 101 is immune antigens, the connection tail 144 may be a monoclonal or polyclonal antibody capable of coupling to a side of the antigen other than a side of the antigen where the capture layer 130 is coupled.

The nanoparticle head 142 and the connection tail 144 can be bonded using various chemicals. For example, the nanoparticle head 142 and the connection tail 144 can be bonded using an ethoxysilane-based hydrocarbon having three to nine carbon atoms. In another example, the nanoparticle head 142 and the connection tail 144 can be bonded using a methoxysilane-based hydrocarbon having two to nine carbon atoms.

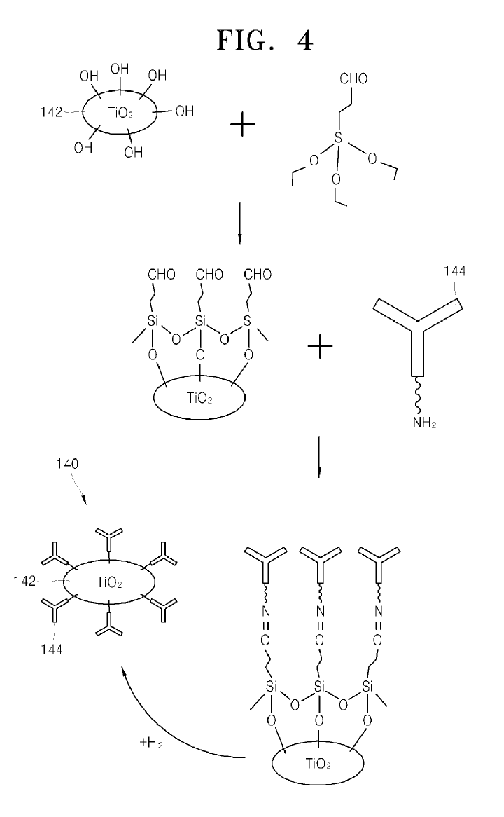

FIG. 4 is a view illustrating a method of fabricating the nano complex 140, according to an embodiment of the present invention.

In the embodiment shown in FIG. 4, TiO₂ is used for forming the nanoparticle heads 142. However, as described above, other materials can be used for forming the nanoparticle heads 142. The nanoparticle heads 142 can be chemically treated, for example, using a Pirahna solution (a 1:1 mixture of sulfuric acid and hydrogen peroxide) to form a sufficient amount of hydroxyl groups on surfaces of the nanoparticle heads 142.

Thereafter, as shown in FIG. 4, the nanoparticle heads 142 are treated in a solvent or a dispersing medium using methoxysilane-based or ethoxysilane-based hydrocarbon having aldehyde groups to allow the hydroxyl groups of the nanoparticle heads 142 to react with the methoxysilane-based or ethoxysilane-based hydrocarbon in order to form the aldehyde groups on the surfaces of the nanoparticle heads 142. The ethoxysilane-based hydrocarbon may have three to nine carbon atoms, preferably four to nine carbon atoms, or more preferably five to nine carbon atoms. The methoxysilane-based hydrocarbon may have two to nine carbon atoms, preferably three to nine carbon atoms, or more preferably four to nine carbon atoms.

The connection tails 144 can be formed of a material having amine groups (—NH₂) that can form a biochemical bond with the target biomaterial 101. The amine groups of the connection tails 144 react with the aldehyde groups of the nanoparticle heads 142 to form —CH═N— bonds. A reductive agent is used to transform the —CH═N— bonds into stable —CH₂—NH— bonds. In this way, the connection tails 144 are respectively bonded to the surfaces of the nanoparticle heads 142 and thus form the nano complex 140.

According to another embodiment of the present invention, a method of detecting a target biomaterial is provided. The method may include: providing a guided mode resonance filter; bringing a capture layer of the guided mode resonance filter into contact with a sample containing a target biomaterial; and bringing a nano complex into contact with the target biomaterial.

The bringing of the capture layer into contact with the sample can be performed before, during, or after the bringing of the nano complex into contact with the target biomaterial. The contact time between the nano complex and the target biomaterial and/or the contact time between the capture layer and the target biomaterial may be in the range of about ten seconds to about two hours according to the kind of the target biomaterial, the kind of the nano complex, temperature, and viscosity. If the nano complex is brought into contact with the target biomaterial by continuously flowing a fluid, the contact time may be a mean residence time (MRT) in a contact region.

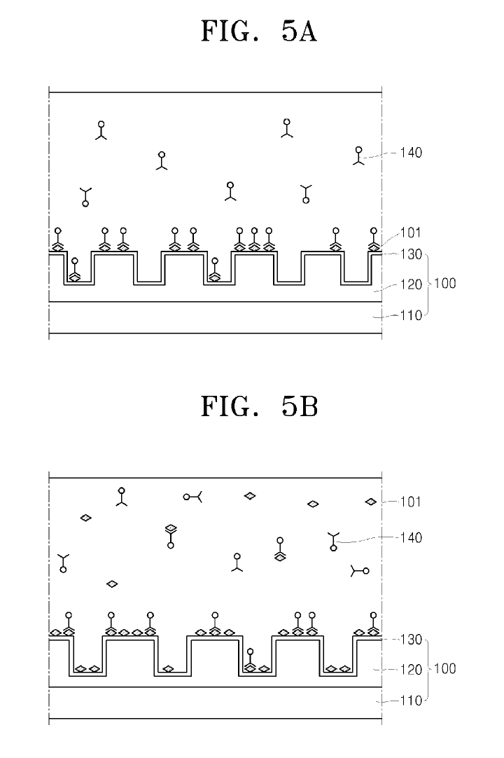

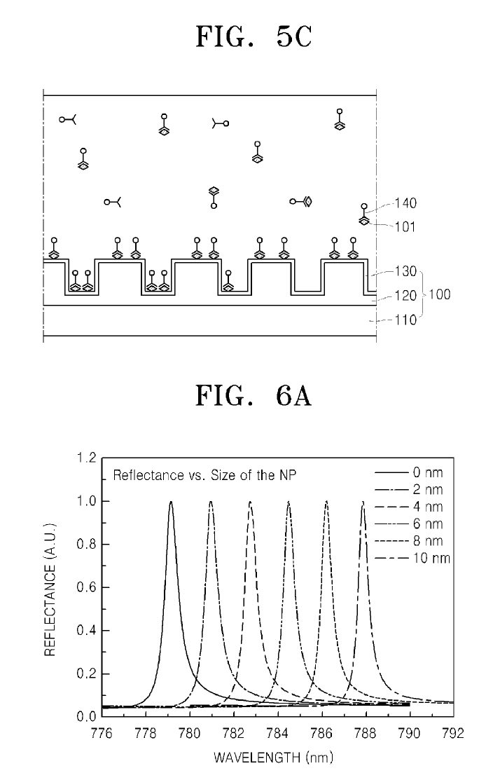

FIG. 5A conceptually illustrates the case where the capture layer 130 of the guided mode resonance filter 100 is brought into contact with a sample containing the target biomaterial 101 before the nano complex 140 is brought into contact with the target biomaterial 101. FIG. 5B conceptually illustrates the case where the capture layer 130 is brought into contact with the sample substantially at the same time when the nano complex 140 is brought into contact with the target biomaterial 101. FIG. 5C conceptually illustrates the case where the capture layer 130 is brought into contact with the sample after the nano complex 140 is brought into contact with the target biomaterial 101.

Referring to FIG. 5A, since the capture layer 130 is brought into contact with the sample containing the target biomaterial 101 before the nano complex 140 is brought into contact with the target biomaterial 101, the capture layer 130 is first covered with the target biomaterial 101. Thereafter, a solvent or dispersing medium in which the nano complex 140 is dissolved or dispersed is allowed to flow on the capture layer 130 in order to couple the nano complex 140 to the target biomaterial 101. In this case, some of the nano complex 140 may remain in the solvent or dispersing medium not being coupled with the target biomaterial 101.

Referring to FIG. 5B, the capture layer 130 is brought into contact with a sample containing the target biomaterial 101 substantially at the same time when the nano complex 140 is brought into contact with the target biomaterial 101. Therefore, combined entities of the nano complex 140 and the target biomaterial 101 are shown on the capture layer 130 and in a solvent or dispersing medium. The target biomaterial 101 may exist both on the surface of the capture layer 130 and in the solvent or dispersing medium. In addition, an uncoupled nano complex 140 may exist in the solvent or dispersing medium.

Referring to FIG. 5C, the capture layer 130 is brought into contact with the sample containing the target biomaterial 101 after the nano complex 140 is brought into contact with the target biomaterial 101. In this case, the concentration of the nano complex 140 may be sufficiently higher than that of the target biomaterial 101. Otherwise, the amount of the target biomaterial 101 coupled to the capture layer 130, without the nano complex 140, can be increased. In this case, the sensitivity of the guided mode resonance filter 100 may decrease.

FIGS. 6A and 6B are graphs showing wavelength peaks for differently-sized nanoparticle heads of a nano complex. FIG. 6A shows wavelength peaks obtained using no nano complex, and nano complexes having 2-nm, 4-nm, 6-nm, 8-nm, and 10-nm nanoparticle heads. FIG. 6B shows wavelength peaks obtained using no nano complex, and nano complexes having 10-nm, 20-nm, 30-nm, 40-nm, 50-nm, 60-nm nanoparticle heads.

Referring to FIGS. 6A and 6B, the displacement of the wavelength peaks increases in proportion to the size of the nanoparticle heads.

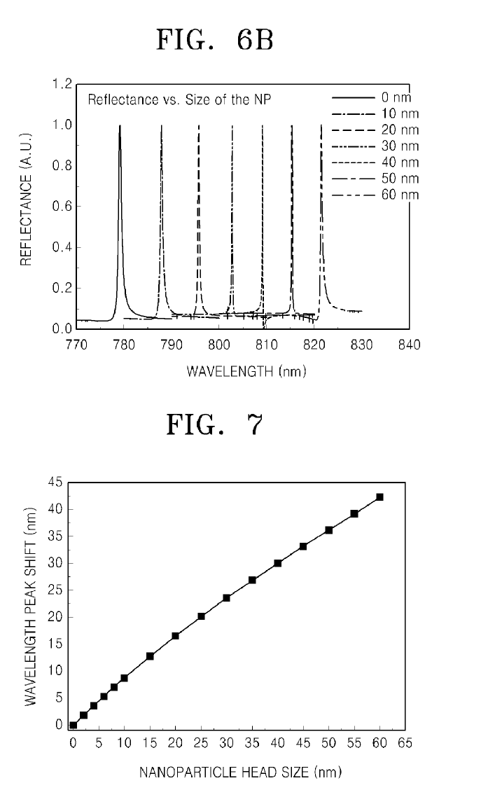

FIG. 7 is a graph illustrating a relationship between wavelength peak displacement and nanoparticle head size. The graph of FIG. 7 is obtained by plotting the wavelength peak displacement with respect to the size of the nanoparticle heads.

Referring to FIG. 7, the wavelength peak displacement is approximately linearly proportional to the nanoparticle head size.

While the present invention has been particularly shown and described with reference to exemplary embodiments thereof, it will be understood by one of ordinary skill in the art that various changes in form and details may be made therein without departing from the spirit and scope of the present invention as defined by the following claims.

1. A target biomaterial detecting kit comprising:
a guided mode resonance filter comprising a substrate transmitting or reflecting light, a grating layer formed on the substrate, and a capture layer formed on the grating layer to capture a target biomaterial; and
a nano complex comprising a nanoparticle head and a connection tail, wherein the nanoparticle head is formed of a metal oxide, a metal nitride, a metal sulfide, a silicon oxide, a silicon nitride, or a silicon sulfide, and
the connection tail is connected to the nanoparticle head and is capable of biochemically coupling to the target biomaterial.

2. The target biomaterial detecting kit of claim 1, wherein the nanoparticle head is formed of a zinc sulfide, TiO₂, an indium tin oxide, an indium zinc oxide, a tantalum oxide, a silicon nitride, or a silicon oxide.

3. The target biomaterial detecting kit of claim 1, wherein the nanoparticle head has a size in a range of about 1 nm to about 60 nm.

4. The target biomaterial detecting kit of claim 1, wherein the connection tail is capable of coupling with the target biomaterial by an antigen-antibody reaction.

5. The target biomaterial detecting kit of claim 1, wherein the connection tail is connected to the nanoparticle head using ethoxysilane-based hydrocarbon having three to nine carbon atoms or methoxysilane-based hydrocarbon having two to nine carbon atoms.

6. A method of detecting a target biomaterial comprising:
providing a guided mode resonance filter, the guided mode resonance filter comprising a substrate transmitting or reflecting light, a grating layer formed on the substrate, and a capture layer formed on the grating layer to capture a target biomaterial;
bringing the capture layer of the guided mode resonance filter into contact with a sample containing a target biomaterial; and
bringing a nano complex into contact with the target biomaterial, wherein the nano complex comprises a nanoparticle head and a connection tail, the nanoparticle head is formed of a metal oxide, a metal nitride, a metal sulfide, a silicon oxide, a silicon nitride, or a silicon sulfide, and
the connection tail is connected to the nanoparticle head and is capable of biochemically coupling to the target biomaterial.

7. The method of claim 6, wherein the bringing of the capture layer into contact with the sample is performed prior to the bringing of the nano complex into contact with the target biomaterial.

8. The method of claim 6, wherein the bringing of the capture layer into contact with the sample is performed simultaneously with the bringing of the nano complex into contact with the target biomaterial.

9. The method of claim 6, wherein the bringing of the capture layer into contact with the sample is performed after the bringing of the nano complex into contact with the target biomaterial.

10. The method of claim 6, wherein the nanoparticle head is formed of a zinc sulfide, TiO₂, an indium tin oxide, an indium zinc oxide, a tantalum oxide, a silicon nitride, or a silicon oxide.

11. The method of claim 6, wherein the nanoparticle head has a size in a range of about 1 nm to about 60 nm.

12. The method of claim 6, wherein the connection tail couples with the target biomaterial by an antigen-antibody reaction.

13. The method of claim 6, wherein the connection tail is connected to the nanoparticle head using ethoxysilane-based hydrocarbon having three to nine carbon atoms or methoxysilane-based hydrocarbon having two to nine carbon atoms.
